(19) United States
(12) Patent Application Publication
(10) Pub. No.: US 2014/0242258 A1
(43) Pub. Date: Aug. 28, 2014

(54) IMPLANTABLE MATERIALS

(71) Applicant: Allergan, Inc., Irvine, CA (US)

(72) Inventors: Alexei Goraltchouk, Rensselaer, NY (US); Jordan Thompson, Chino Hills, CA (US); Miram M. Abiad, Costa Mesa, CA (US); Kevin A. Ma, Scotts Valley, CA (US)

(73) Assignee: Allergan, Inc., Irvine, CA (US)

(21) Appl. No.: 14/271,292

(22) Filed: May 6, 2014

Related U.S. Application Data
(62) Division of application No. 13/213,925, filed on Aug. 19, 2011, now abandoned.
(60) Provisional application No. 61/375,686, filed on Aug. 20, 2010.

(51) Int. Cl. A61L 27/00

(52) U.S. Cl. CPC A61L 27/00
USPC 427/2.24

(57) ABSTRACT

A textured breast implant is provided which generally includes a fluid fillable elastomeric shell having a texture defined by struts, for example, hollow struts, defining interconnected open cells. Methods of making the texture include applying a silicone dispersion to a base material and removing the base material from the coating to form a silicone-based structure comprising struts defining interconnected open cells, said struts including internal surfaces defining cavities within the struts. The method may further include the step of contacting the silicone based structure having cavities with a silicone dispersion to cause the silicone to enter and fill the cavities.

This application is a divisional of U.S. patent application Ser. No. 13/213,925, filed Aug. 19, 2011, which claims priority to U.S. Patent Application No. 61/375,686, filed Aug. 20, 2010, the entire disclosure of each of these applications being incorporated herein by this reference.

### BACKGROUND

The present invention generally relates to medical implants and more specifically relates to foam-like materials suitable for implantation in a mammal.

Prostheses or implants for augmentation and/or reconstruction of the human body are well known. Capsular contracture is a complication associated with surgical implantation of prostheses, particularly with soft implants, and even more particularly, though certainly not exclusively, with fluid-filled breast implants.

Capsular contracture is believed to be a result of the immune system response to the presence of a foreign material in the body. A normal response of the body to the presence of a newly implanted object, for example a breast implant, is to form a capsule of tissue, primarily collagen fibers, around the implant. Capsular contracture occurs when the capsule begins to contract and squeeze the implant. This contracture can be discomforting or even extremely painful, and can cause distortion of the appearance of the augmented or reconstructed breast. The exact cause of contracture is not known. However, some factors may include bacterial contamination of the implant prior to placement, submuscular versus subglandular placement, and smooth surface implants versus textured surface implants, and bleeding or trauma to the area.

Surface texturing has been shown to reduce capsular contracture when compared to what are known as “smooth” surface implants.

There is still a need for a more optimal surface textured implant that further reduces the potential for capsular contracture. The present invention addressed this need.

### SUMMARY OF THE INVENTION

Accordingly, the present invention provides a method of making a material suitable for implantation in a mammal. The method generally comprises the steps of providing a base member including a porous surface defined by interconnected pores and contacting the base member with a silicone-based fluid material in a manner to cause the fluid material to enter the pores. In one embodiment, a vacuum is applied to the base member to draw the fluid material into and/or through the pores. The method may comprise the steps of removing excess fluid material from the base member to obtain a coating of the fluid material on the porous surface, and allowing the coating to set to form a silicone-based structure suitable for implantation in a mammal. The removal process can be obtained using an airknife to blow away the excess material, and/or squeezing out the excess material, and/or using suction to remove the excess material. The silicone-based structure includes a porous surface, having interconnected cells, the porous surface substantially identically conforming to the porous surface of the base member.

In one aspect of the invention, the base material is a material which can be degraded or otherwise removed from within the coating without substantially affecting the coating structure. In some embodiments, the base material is a substantially biodegradable material. The base material may be polyurethane, for example, polyurethane foam. Alternatively, the base member is melamine, for example, melamine foam. Other base member materials are also contemplated and include, for example, foams made from polyethylene, polyethylene vinyl acetate, polystyrene, polyvinyl alcohol, or generally a polyolefin, polyester, polyether, polyamide, polysaccharide, a material which contains aromatic or aliphatic structures in the backbone, as functionalities, crosslinkers or pendant groups, or a copolymer, terpolymer or quarternaly polymer thereof. Alternatively the material may be a composite of one or more aforementioned materials. In another embodiment of the invention the base material can be a metal, for example a metal foam, a ceramic, or a composite material.

The silicone-based fluid material may comprise a dispersion, for example, a silicone dispersion, solution, emulsion or mixture. The silicone-based fluid material may be a solution of a room temperature vulcanizing (RTV) or a high temperature vulcanizing (HTV) silicone from about 0.1-95 wt %, for example, about 1-40 wt %, for example, about 30 wt %. In an exemplary embodiment, the silicone-based fluid material is a high temperature vulcanizing (HTV) platinum-cured silicone dispersion in xylene.

In another aspect of the invention, the base member, or at least a portion thereof, is removed from the silicone-based structure. In one embodiment, substantially all of the base material is removed, such that a product is obtained which comprises or consists of material that is substantially entirely pure silicone, for example, a porous, cellular silicone foam. The step of removing may comprise, for example, contacting the base member with a solution capable of dissolving the base member. For example, in an embodiment of the invention in which the base member is polyurethane foam, the step of removing may comprise contacting the base member with a hydrogen peroxide solution. In other embodiments of the invention, the base material may be degraded by exposure to UV light, heat, oxidative agents, a base such as sodium hydroxide, or an acid such as phosphoric acid or a combination thereof. The material may be exhaustively removed further by a secondary process such as solvent leach or vacuum.

In another aspect of the invention, a material suitable for implantation in a mammal is provided. The material comprises a porous, cellular member comprising a silicone-based structure. The silicone-based structure has a topography, for example, a pore size, shape and interconnectivity, substantially identical to that of a polyurethane foam. This material may be made by the processes in accordance with methods of the invention, as described herein.

In yet another aspect of the invention, a method of making a material suitable for implantation in a mammal is provided which generally comprises providing a base member comprising a degradable foam and including a porous surface defined by interconnected pores, and coating the base member with a substantially non-biodegradable polymeric material to obtain a substantially non-biodegradable polymeric structure suitable for implantation in a mammal. More specifically, the method includes contacting the base member with a fluid precursor of the substantially non-biodegradable polymeric material in a manner to cause the fluid precursor to enter the pores, removing excess fluid precursor material to obtain a coating of the fluid precursor on the base member, and allowing the coating to set to form the substantially non-biodegradable polymeric structure. The resulting structure includes a porous surface substantially identically conforming to the porous surface of the base member.

In yet another aspect of the invention, a method is provided which generally comprises providing a base member including a porous surface defined by interconnected pores, contacting the base member with a first material, allowing the first material to set to form a first material coating on the base member, contacting the first material coating with a second material different from the first material and allowing the second material to set to form a layered polymeric structure suitable for implantation in a mammal. The resulting layered polymeric structure includes a porous surface substantially identically conforming to the porous surface of the base member. In an exemplary embodiment, the first material is a fluorinated polyolefin material and the second material is a silicone dispersion.

In yet another aspect of the invention, a method of making a material is provided, the method generally comprising the steps of providing a base member having a surface defined by a geometry including interconnected pores, forming a first coating on the surface of the base member material, the first coating being selected from the group of materials consisting of polystyrene, polyethylene-co-vinyl acetate, and poly (styrene-co-butadiene-co-styrene), and removing the polymeric base member by contacting the base material with a material that will cause the base member to be removed from the first coating without causing any substantial degradation of the first coating. Next, a silicone-based fluid material is applied to the first coating which now has the base member removed therefrom, and cured to form a silicone coating on the first coating. The first coating is then removed from the silicone coating, for example, by dissolving away the first coating from the silicone, thereby forming a silicone foam-like material.

Each and every feature described herein, and each and every combination of two or more of such features, is included within the scope of the present invention provided that the features included in such a combination are not mutually inconsistent.

### BRIEF DESCRIPTION OF THE DRAWINGS

The present invention may be more clearly understood and certain aspects and advantages thereof better appreciated with reference to the following Detailed Description when considered with the accompanying Drawings of which:

FIG. 1 is an SEM micrograph of a implantable material made in accordance with a method of the invention.

FIGS. 2-9 are images of other materials that can be useful as base materials in accordance with different embodiments of the invention.

FIG. 10 is a SEM image of a base material, specifically a polyurethane foam material at 500× magnification, useful in the manufacturing of some of the silicone-based materials of the present invention.

FIG. 11 is a polyurethane foam material such as shown in FIG. 10, now coated in silicone in accordance with certain embodiments of the invention, at 500× magnification.

FIG. 12 is an image similar to that shown in FIG. 11, except a more viscous silicone has been applied to the polyurethane, resulting in more silicone being deposited, relative to that shown in FIG. 11.

FIG. 13 is a substantially hollow, foam-like silicone material (shown at 400× magnification) in accordance with the invention, after a polyurethane material has been removed from (e.g. leached out of) the silicone coating, leaving voids or hollows in the struts and cell walls of the foam-like silicone material.

FIG. 14 is a foam-like silicone material (shown at 400× magnification), in accordance with another aspect of the invention, after a silicone material such as shown in FIG. 13 has been contacted with additional silicone, thus filling the voids left by the removed polyurethane material.

FIGS. 15A-15H are SEM images of various silicone-based foam-like materials in accordance with embodiments of the present invention.

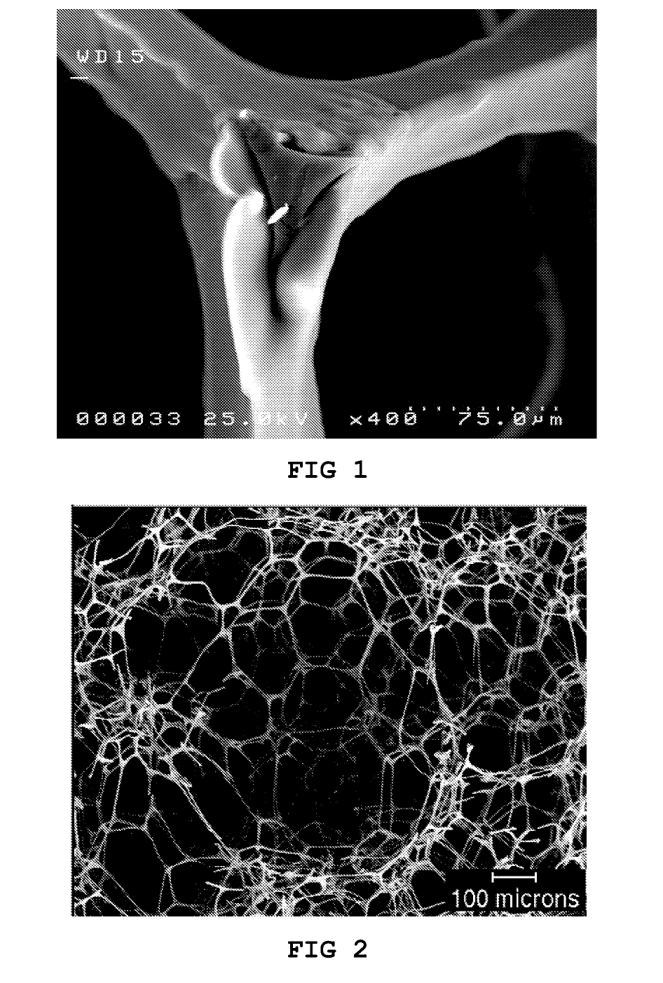

### DETAILED DESCRIPTION

The present invention generally pertains to foam-like materials, for example, biocompatible, foam-like materials, for example, implantable materials and methods of forming same. The materials are useful for a variety of purposes, including, but not limited to, use in medical environments.

In one aspect of the invention, methods are provided for making an implantable material that is substantially biologically inert and/or substantially non-biodegradable, which has a structure, for example, a microstructure, similar or substantially identical to that of a foam of a different material. The different material may be, or may not be, a biologically inert or non-biodegradable material.

In a specific embodiment, the foam-like materials are substantially entirely comprised of silicone yet have the topographical structure of a non-silicone material, for example, a polyurethane foam.

For example, a material in accordance with one embodiment is a flexible, soft, silicone-based foam-like material having substantially the same or substantially identical geometry of a polyurethane foam, but with the chemical inertness and biocompatibility of a silicone.

For example, a method for making a foam-like material substantially entirely comprised of silicone generally comprises the steps of providing a base member, for example a polyurethane base member including a porous surface defined by interconnected pores and contacting the base member with a silicone-based fluid material. The contacting step is done in a manner to ensure coating of the base member with the silicone material in a manner to cause the fluid material to enter the pores and conformally coat the surfaces of the base material. In some embodiments, a vacuum may be applied to the base material in order to facilitate the contacting step. Excess fluid material may be removed from the base member to obtain a fine coating of the fluid material on the porous surface of the base material. The silicone-based coating is allowed to set. The coating steps may be repeated once, twice, three or more times, for example, up to 1000 times, until a desired thickness and/or final foam density is achieved. In some embodiments, the underlying polyurethane material may be removed from the coating structure. For example, the polyurethane is contacted with a dissolvent, dimethyl sulfoxide, or a degradant such as hydrogen peroxide or hydrochloric acid, followed by a dissolvent such as dimethyl sulfoxide of dimethyl formamide or acetone. The resulting silicone-based material is flexible and biocompatible and includes a porous surface substantially identically conforming to the geometry of a porous surface of a polyurethane foam.

It is to be appreciated that for a base material other than polyurethane, said base material can be removed by a solvent or other means, known to those of skill in the art, suitable for removing the base material from the coating without substantially altering or affecting the coating structure.

The base material may have a pore size of about 100-1000 μm (RSD, i.e. relative standard deviation, of about 0.01-1000); an interconnection size of about 30-700 μm (RSD of 0.01-100%); interconnections per pore of about 2-20 (RSD of 0.01-50%); and an average pore to interconnection size ratio of about 3-99%.

In some embodiments, the base material has a pore size of about 300-700 μm (RSD of 1-40%); an interconnection size of about 100-300 μm (RSD of 1-40%); interconnections per pore of about 3-10 (RSD of 1-25%) and an average pore to interconnection size ratio of about 10% to about 99%.

In an exemplary embodiment, the base member comprises a material, for example, polyurethane or other suitable material, having a pore size of 472+/−61 μm (RSD=13%), interconnection size: 206+/−60 μm (RSD=29%), interconnections per pore: 9.6+/−1.8 (RSD=19%), Pore to interconnection size ratio of about 44%.

The base material may be a foam with between about 20 ppi to about 150 ppi, for example, between about 60 ppi to 100 ppi, for example, about 80 ppi. In a specific embodiment, the base material is a polyurethane foam with about 100 ppi.

The base material may have a thickness of between about 1 mm to about 5 mm. In a specific embodiment, the base material has a thickness of about 3 mm.

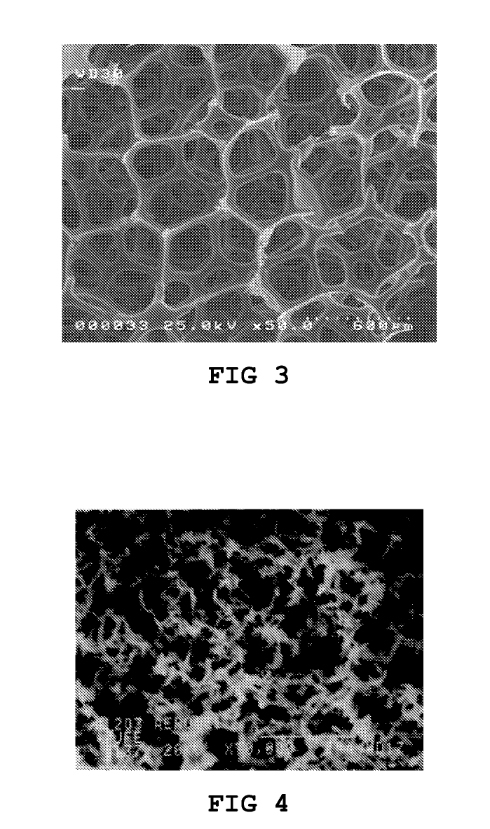

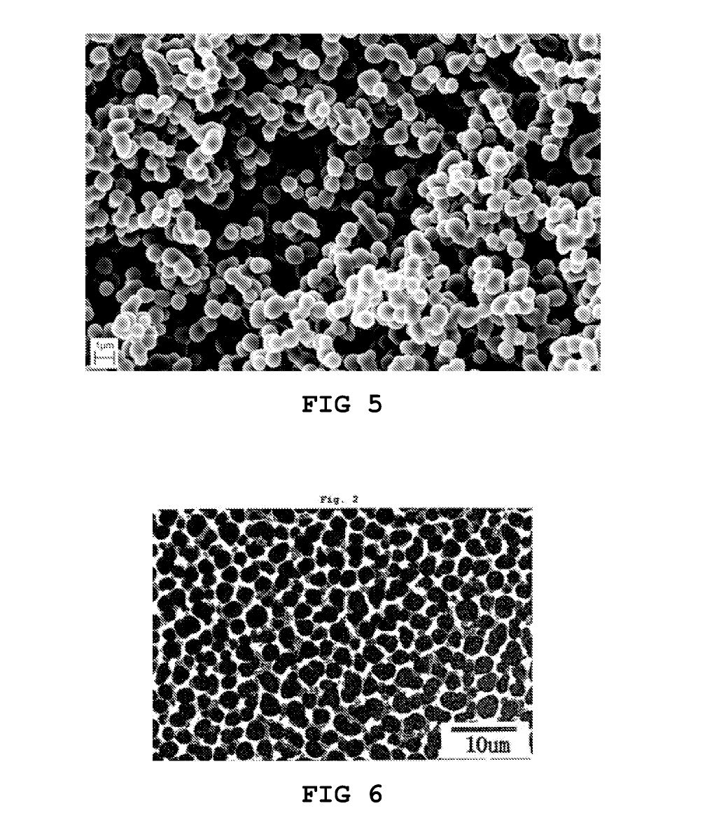

The base member may comprise any suitable porous material having the desired surface structure. Alternative to polyurethane, the base member may comprise melamine, for example, melamine foam. FIG. 2 is an SEM micrograph of a melamine foam having a topography defined by highly interconnected, open pores. Other base member materials useful in the methods of the invention are also contemplated and include, for example, polyethylene foam, Styrofoam, or general polyolefin foams, polysaccharide foams, polyamide foams, polyacrylate foams, metal and/or ceramic foams, and combinations thereof.

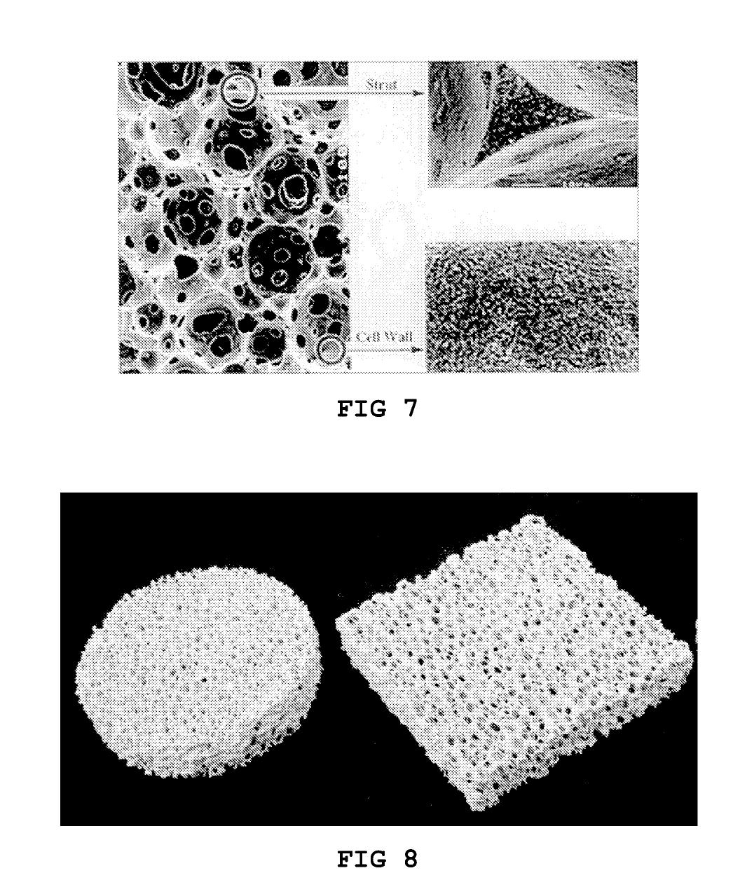

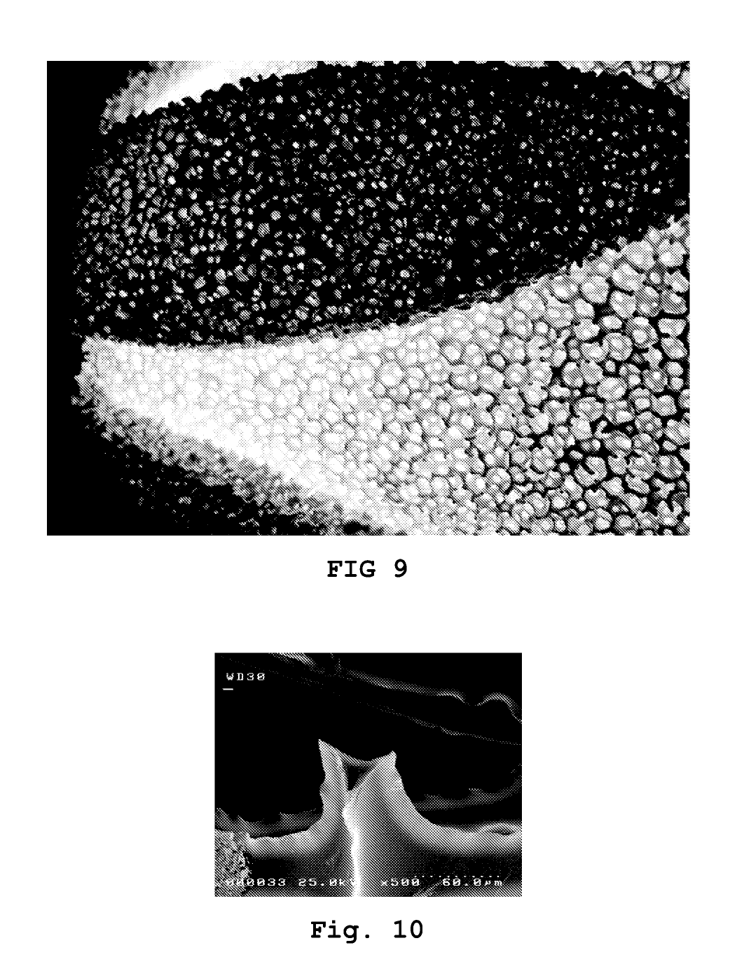

Porous surfaces of base member materials having a variety of surface geometries useful in accordance with various embodiments of the invention are shown in FIGS. 2-9. More specifically, FIG. 2 is a SEM micrograph of a melamine foam. FIG. 3 is a SEM image of a polyurethane foam; FIG. 4 is an alumina aerogel foam; FIG. 5 is another aerogel, for example, silica aerogel foam; FIG. 6 is a silica foam; FIG. 7 is a HiP foam; FIG. 8 is a magnesium ceramic foam; and FIG. 9 is another ceramic foam.

In an exemplary embodiment, the silicone-based fluid material may comprise a dispersion, for example, a silicone dispersion, for example, a room temperature vulcanizing (RTV) or a high temperature vulcanizing (HTV) silicone dispersion. In an exemplary embodiment, the silicone-based fluid material is a high temperature vulcanizing (HTV) platinum-cured silicone dispersion in xylene or chloroform. The silicone-based fluid material may be commercially available HTV silicone such as NuSil MED 4714. Percent solids in the coating dispersion are generally between about 15% to about 40%, for example, about 15%.

In some embodiments, the non-biodegradable polymeric structure, for example, the silicone structure, may have a weight of at least about 3 times, for example, at least 5 times for example, up to 10 times or more, the weight of the base member coated thereby. In some embodiments, the silicone structure has a weight of between about 3 times and about 10 times the weight of the based member coated thereby and is formed from only a single coating of the silicone dispersion, for example, a single contacting step. For example, the percent pickup of some of the present methods is between about 300% and about 1000%, where “percent pickup” is defined as the % weight gain of the coated material verses the starting weight of the base material. Therefore a 100% pickup of a coating would be where the coated material is the same weight as the initial base member. For example, if the base member is 3 grams of polyurethane and the cured silicone coating on the base member is 3 grams of silicone, 100% pickup has been achieved.

In some methods of the present invention, up to 1000% pickup is achieved. For example, a 70 mm diameter round of polyurethane having a weight of 0.3 g, may be coated with silicone in accordance with the present methods with a resulting silicone coated polyurethane having a weight of approximately 3.3 grams, i.e. 1000% pickup. In some embodiments, 300% up to 1000% pickup is achieved using only a single coating step in accordance with the methods of the present invention.

Alternatives to silicone-based polymers are also contemplated. For example, any implantable material that can be cured by crosslinking, thermoplastics that set by change in temperature, material that set by removal of solvents or any elastomer that cures or sets by any known mechanism, can be used. It is further contemplated that other implantable materials useful in accordance with the invention include suitable metals or ceramics.

In another aspect of the invention, methods are provided for making porous materials, for example, flexible, porous silicone-based materials, for example, foam-like materials made substantially entirely of silicone. In one embodiment, a method of making a material is provided comprising the steps of providing a base member including a porous surface defined by interconnected pores, and contacting the base member with a fluid first material, for example, a non-silicone based material, in a manner to cause the fluid first material to enter the pores. The first material is allowed to set to form a first material coating on the base member and the first material coating is contacted with a fluid silicone-based material, for example a silicone dispersion. The fluid silicone-based material is allowed to set to form a silicone-based material coating on the first material coating, thereby forming a layered polymeric structure defined by a surface substantially identically conforming to the surface of the base member. In a specific embodiment, the first material is a fluorinated polyolefin material.

In yet another aspect of the invention, a method of making a material suitable for implantation in a mammal is provided which generally comprises providing a base member comprising a degradable foam and including a porous surface defined by interconnected pores, and coating the base member with a substantially non-biodegradable polymeric material to obtain a substantially non-biodegradable polymeric structure suitable for implantation in a mammal. For example, the base member may comprise a polyurethane foam. The substantially non-biodegradable polymeric material can be any suitable biocompatible polymer and may be selected from a list of highly impermeable systems, such as but not limited to, fluorinated polyolefins, to prevent diffusion of chemical entities which may facilitate the degradation of polyurethane. Alternatively the fluorinated polyolefin can be coated as a base layer, prior to the final application of the silicone to act as a barrier layer.

In another embodiment of this invention, the base member of a preferred geometry, that is not dissolvable (for example, a crosslinked polymer having a porous surface) may be coated by a robust but dissolvable material, such as, for example, a foam material selected from the group of materials consisting of polystyrene, polyethylene-co-vinyl acetate, and poly(styrene-co-butadiene-co-styrene). The base member, e.g. the non-dissolvable foam, can then be removed from the dissolvable material coating, for example, degraded by relatively aggressive means, for example, by acid digestion in 37% HCl, leaving the robust but dissolvable material behind. An implantable material of interest, for example, a silicone-based fluid material, is deposited on the robust but dissolvable foam, for example, using the methods described elsewhere herein. The silicone-based fluid material may be in the form of a dispersion having a solvent system that does not dissolve the robust polymer. The silicone is allowed to set or cure, and the robust material is then dissolved out by means which does not affect the material of interest (e.g. silicone), for example, by dissolution in acetone in the case of polystyrene. In this case, the material of interest is not subjected to aggressive conditions used to dissolve the original foam.

The present invention also provides a silicone-based foam-like material suitable for implantation, wherein the material generally comprises a porous silicone-based structure including struts defining interconnected cells. The material may be substantially entirely silicone yet have the configuration of a polyurethane foam.

In some embodiments, the struts are substantially hollow, for example, the struts which define the pores of the foam-like material include internal surfaces defining cavities within the struts. This structure may be made by some of the processes described elsewhere herein. The cavities within the struts are negative spaces left behind after removal of a base foam material from a conformal coating of silicone.

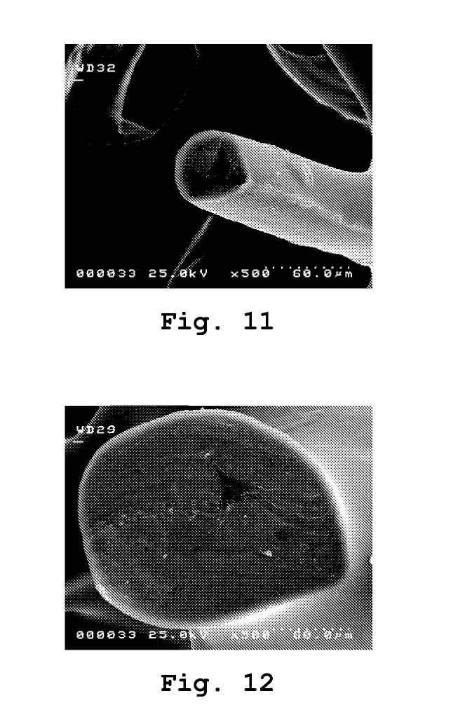

For example, FIG. 10 is an SEM image (500X) of polyurethane foam, useful as a base material in accordance with the invention. FIG. 11 shows an image of a polyurethane strut having a relatively thin coating of silicone in accordance with embodiments of the invention. A strut of the composite material has been cut for this image in order to reveal the base material (polyurethane) within a silicone coating. FIG. 12 is a similar view of a similar silicone-based, foam-like material, with a relatively thicker coating of silicone. The thicker coating may be accomplished by using multiple coatings of a relatively low viscosity silicone dispersion or by a single coating of a relatively thicker, more viscous silicone dispersion.

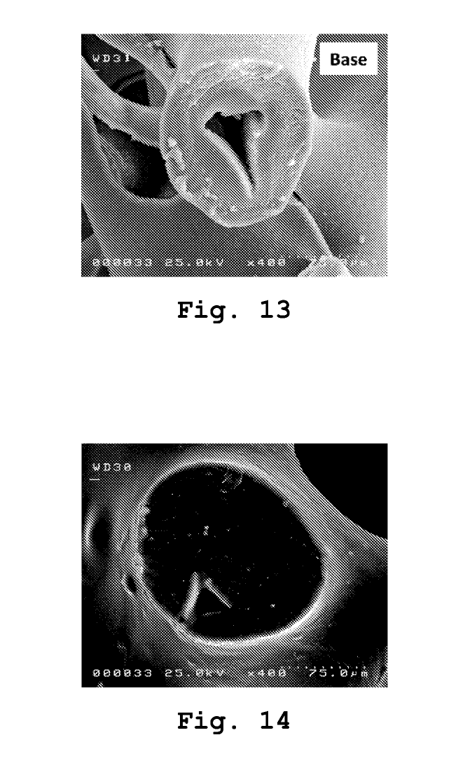

Turning now to FIG. 13, and SEM image of a similar silicone-based, foam-like material such as shown in FIG. 11, is shown, now after removal of foam base material from a silicone coating. The silicone structure, in accordance with this aspect of the invention, thus includes struts defining interconnected cells, and the struts include internal hollows, cavities or voids left behind by the removed base foam. Advantageously, this silicone, foam-like structure may be substantially entirely comprised of, or essentially consist of, silicone, but has a structure closely similar to a true polyurethane foam.

In yet other embodiments, a silicone-based structure may be provided which include struts defining interconnected cells, which are not hollow, but substantially solid. For example, the structure in accordance with this embodiment may be made by the filling in the hollows or cavities left behind by the removed base foam.

For example, FIG. 14 shows a leached silicone-based foam-like material (as in FIG. 3) that has now been contacted with a silicone dispersion, thereby substantially filling in the voids left behind by the removed base material. The material may have a reduced pore size and interconnection diameter in comparison to the base foam, for example, an increased volume to void space. For example, the pore size of the resulting silicone foam-like material may be between about 0.1% to about 100% reduced relative to the base foam which was used to form the silicone foam-like material. The interconnection diameter may range from about 0.1% to 80% of the initial base foam material. In a specific embodiment, the interconnection diameter is about 30% to 50% of the initial base foam material interconnection diameter.

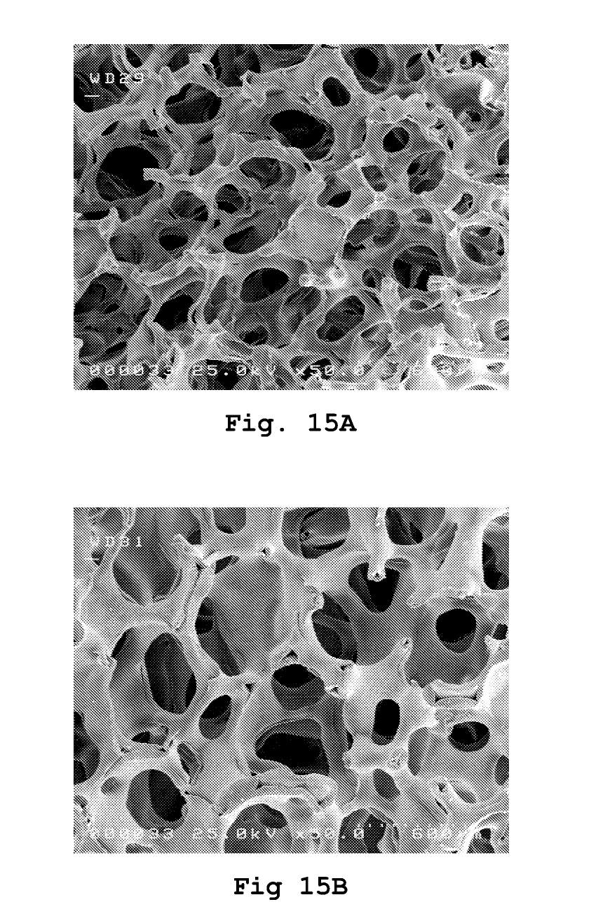

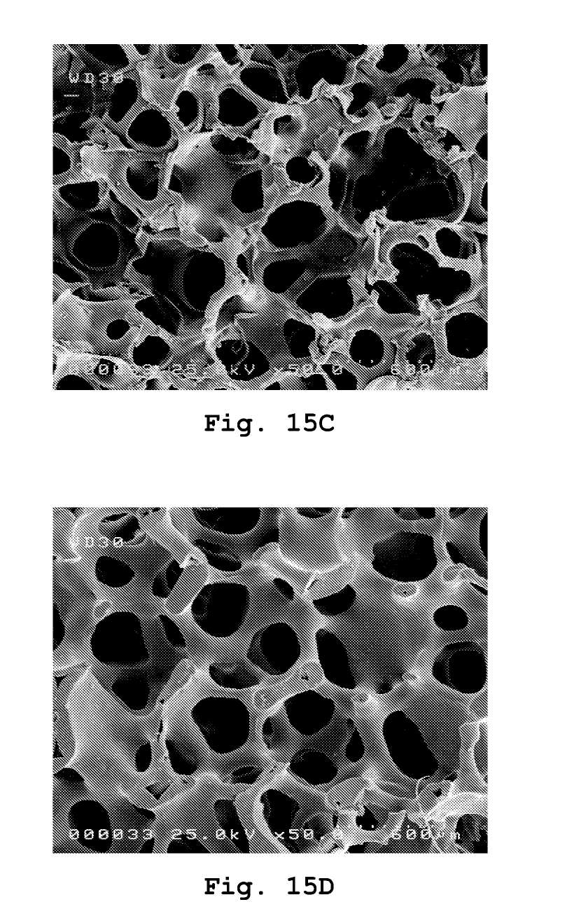

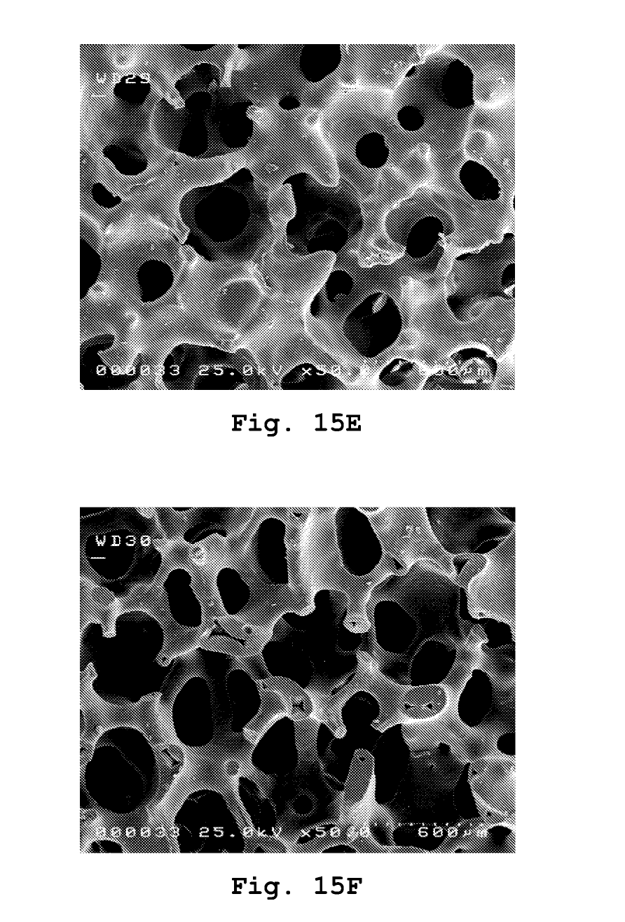

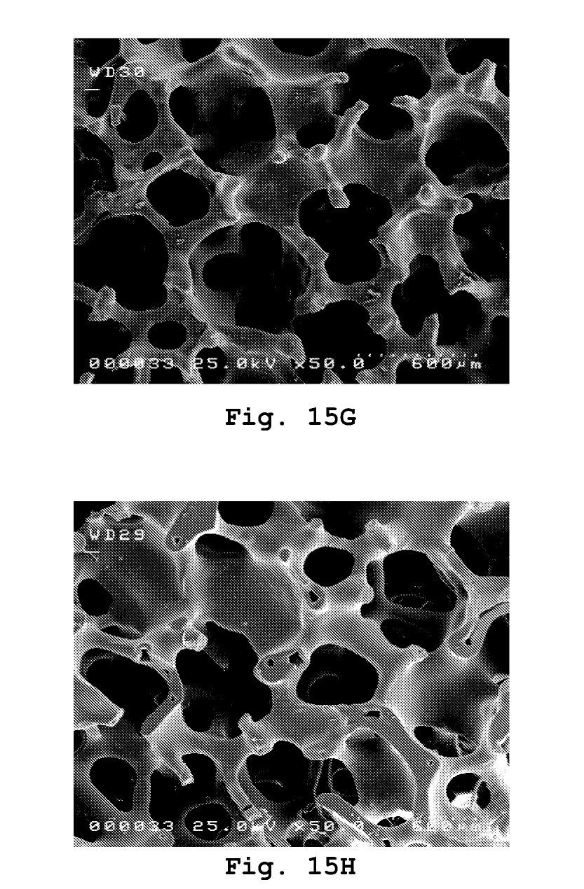

FIGS. 15A-15H are SEM images of various materials made in accordance with methods of the present invention. More specifically, FIGS. 15A-15B are SEM images of top and cross-sectional views, respectively, of a silicone-based material made in accordance with the invention; FIGS. 15C and 15D are SEM images of top and cross-sectional views, respectively, of another silicone-based material (strengthened once with 10% MED 4815 in Xylene); FIGS. 15E and 15F are SEM images of top and cross-sectional views, respectively, of another silicone-based material (strengthened twice with 10% MED 4815 in Xylene; FIGS. 15G and 15H are SEM images of top and cross-sectional views, respectively, of another silicone-based material (strengthened thrice with 10% MED 4815 in Xylene, made in accordance with the invention.

### Example 1

A polyurethane open celled foam is coated according to the current invention using a solution of Silicone HTV 30% w/v, by either dipping the polyurethane foam in the solution, casting the solution on a sheet of polyurethane or spraying the solution in excess over the sheet of polyurethane. The excess solution is removed by squeezing out the foam, or by vacuum at between about 20 in. Hg to about 40 in. Hg, or higher, which may be applied in any suitable manner, for example, through a Buchner funnel at the bottom of the foam (in the case of casting the solution over the foam) or by blowing air over the foam as in the case of an air-knife, or in combination of any of the aforementioned. Air pressure may be applied with a pressure in a range of about 20 to about 100 psi. The foam is then devolitilized in vacuum or by application of mild heat in the case of HTV, such that the solvent is removed, but the HTV is not cured. This can be achieved in the application of the air current during the previous step (the air may or may not be heated). Finally the coated foam is cured and the coating layer is affixed unto the foam. Curing is done at a suitable curing time and temperature, for example, for about 60 minutes at a temperature between about 120° to about 150° C., depending on the materials used. The aforementioned coating, removing, devolitizing and curing process may be repeated one or more times, for example, up to 5 times for example, up to 10 times, for example, up to 20 times, for example, up to 50 times, for example, up to 200 times, for example, up to 500 times, for example, up to 1000 times, to achieve various builds and/or final pore densities. The polyurethane may then be completely removed from the center of the silicone structure by digestion in hydrogen peroxide/water solution with or without the presence of metal ions and with or without heating. Alternatively the polyurethane foam can be degraded out by 37% HCl digestion for 1-5 minutes, with vigorous agitation and air removal to facilitate the uniform digestion of the polyurethane, and a subsequent DMSO wash to remove the remnant degradants which are not soluble in the 37% HCl. The degradation/leaching steps can be repeated 1-20 times to achieve various levels of purity. The resulting material is a substantially pure silicone foam useful as a surgical implant.

### Example 2

A sheet polyurethane open celled foam (20×20 cm) is placed in a container the bottom of which is a fine grate. Vacuum is applied to the bottom of the grate to pull air through the top of the foam into the foam and finally through the grate and out. A solution of about 20% HTV (platinum cured) in chloroform is cast over the foam and pulled through the foam by the vacuum, a jet of air is applied to the foam through an air-knife to remove any remaining solution droplets that are trapped in the foam to clean out the pores. The foam is then devolitized in vacuum at about room temperature for 2 hours. The devolitized foam is finally cured at 120° C. for 1 hour. The process is repeated 3 times. The resulting foam is an open celled polyurethane base foam, conformally coated by an approximately 50 μm layer of silicone.

### Example 3

An implantable material is produced substantially in accordance with Example 2, except that instead of a polyurethane foam, a melamine foam is used as the base member. In addition, the base material is not removed from the silicone foam. The resulting implantable material comprises a highly porous, open celled structure having a melamine base and a silicone overcoat.

### Example 4

The silicone foam of Example 1 is produced as a flexible sheet. The sheet is cut and laminated to form a front surface of a breast implant. The front surface of the breast implant has a surface texture substantially identical to a surface texture of a polyurethane foam, but is substantially pure silicone.

### Example 5

A 20×20 cm sheet polyurethane open-celled foam of 100 ppi and a thickness of about 3 mm, is placed on a fine grate. Vacuum (about 29 in. Hg) is applied to the bottom of the grate to pull air through the foam and grate. A solution of about 15% HTV Silicone (NuSil MED 4714) (platinum cured) in Xylene is cast over the foam and pulled through the foam by the vacuum. A jet of air (about 100 psi) is applied to the foam through an air-knife to remove any remaining solution droplets that are trapped in the foam and to clean out the pores. The coated foam is then devolitized in vacuum at about room temperature for 2 hours. The devolitized foam is finally cured at 126° C. for 1 hour. The above described process is repeated times. The cured silicone-coated foam is contacted with dimethylsulfoxide and is placed in a shaker and agitated at room temperature overnight to remove the polyurethane. The resulting structure is an porous open-celled silicone member having a structure closely matching the original polyurethane foam and made up of hollow struts (hollows formed by the removed polyurethane) having a substantially uniform wall thickness of about 80 μm.

### Example 6

A 20×20 cm sheet polyurethane open-celled foam of 80 ppi and a thickness of about 3 mm, is placed on a fine grate. Vacuum (about 29 in. Hg) is applied to the bottom of the grate to pull air through the foam and grate. A solution of fluorinated polyolefin is applied to the foam and allowed to set to form a fine coating of about 50 microns in thickness on the foam. A solution of about 15% HTV Silicone (NuSil MED 4714) (platinum cured) in Xylene is cast over the fluorinated polyolefin coated foam and pulled through the foam by the vacuum. A jet of air (about 100 psi) is applied to the foam through an air-knife to remove any remaining solution droplets that are trapped in the foam and to clean out the pores. The coated foam is then devolitized in vacuum at about room temperature for 2 hours. The devolitized foam is finally cured at 126° C. for 1 hour. The step of applying a solution of silicone dispersion is repeated until a cured silicone coating thickness of about 100 microns is achieved.

### Example 7

A porous open-celled silicone member having hollow struts is made as described in Example 5. This foam-like silicone member is placed on a grate and is contacted with a silicone dispersion during application of a vacuum. The silicone dispersion is allowed to devolitize and the silicone dispersion application may be repeated, for example, up to five or more times. The resulting structure is a biocompatible, non-biodegradable foam-like material that has a structure, flexibility and/or elasticity quite similar to a biodegradable polyurethane foam.

### Example 8

The porous open-celled silicone member having hollow struts is made as described in Example 7. This foam-like silicone member is then layered onto a smooth breast implant shell using a suitable biocompatible adhesive. The implant has a reduced likelihood of promoting capsular contracture when implanted in a patient, relative to an implant having a smooth shell without the open-celled silicone member adhered thereto.

While this invention has been described with respect to various specific examples and embodiments, it is to be understood that the invention is not limited thereto and that it can be variously practiced within the scope of the invention.

What is claimed is:

1. A method of making a material suitable for implantation in a mammal, the method comprising:
providing a base member including a porous surface defined by interconnected pores;
contacting the base member with a fluid silicone-based material in a manner to cause the fluid material to enter the pores;
applying a vacuum to the base member to draw the fluid material into the pores;
removing excess fluid material from the base member to obtain a coating of the fluid material on the porous surface; and
allowing the coating to set;
removing the base material from the coating after allowing the coating to set to form a silicone-based structure comprising struts defining interconnected open cells, said struts including internal surfaces defining cavities within the struts.

2. The method of claim 1 wherein the base member is polyurethane foam.

3. The method of claim 1 wherein the coating has a thickness of between about 10 microns and about 100 microns.

4. The method of claim 1 wherein the coating has a thickness of about 50 microns.

5. The method of claim 1 wherein the material is a component of a breast implant.

6. The method of claim 1 further comprising contacting the silicone based structure having cavities within the struts with a silicone dispersion to cause the silicone to enter and fill the cavities.

7. The method of claim 1 wherein the material has about 100 pores per inch (ppi).

9. The method of claim 1 wherein the material has a thickness of between about 1 mm and about 5 mm.

10. The method of claim 1 wherein the material has a thickness of about 3 mm.

11. The method of claim 1 wherein the vacuum is applied at about 20 to about 40 Hg negative pressure.

12. The method of claim 1 wherein the vacuum is applied at about 29 Hg or greater negative pressure.
